(19) United States
(12) Patent Application Publication
(10) Pub. No.: US 2024/0316349 A1
(43) Pub. Date: Sep. 26, 2024

(54) SYSTEMS AND METHODS FOR MANAGING WIRELESS COMMUNICATION WITH IMPLANTED MEDICAL DEVICES

(71) Applicant: The Alfred E. Mann Foundation for Scientific Research, Valencia, CA (US)

(72) Inventors: Hitesh HOTLANI, Santa Clarita, CA (US); Brian SHELTON, Ventura, CA (US)

(21) Appl. No.: 18/612,858

(22) Filed: Mar. 21, 2024

Related U.S. Application Data
(60) Provisional application No. 63/492,123, filed on Mar. 24, 2023.

(51) Int. Cl. A61N 1/372; G16H 40/67

(52) U.S. Cl. CPC A61N 1/37223; A61N 1/37235; G16H 40/67

(57) ABSTRACT

The disclosure provides systems and methods for managing wireless communication with implanted medical devices. In some aspects, such systems and methods comprise or utilize an implanted medical device (“IMD”); and a first external device, configured to wirelessly communicate with the IMD; wherein the first external device is configured to detect a second external device that is capable of wirelessly communicating with the IMD. In some aspects, the first external device may be configured to terminate a wireless connection with the IMD when various predetermined conditions occur, e.g., allowing for the IMD to connect to the second external device.

### CROSS-REFERENCE TO RELATED APPLICATION

This application claims priority to U.S. Provisional Application No. 63/492,123, filed Mar. 24, 2023, the contents of which is hereby incorporated in its entirety.

### BACKGROUND

Implantable medical devices (“IMDs”) are utilized in modern healthcare to treat a variety of medical conditions. For example, IMDs such as implantable pulse generators (“IPGs”), pacemakers, and defibrillators can facilitate management of a wide range of ailments, including, but not limited to, obstructive sleep apnea (“OSA”), cardiac arrhythmia, and neurological diseases. Patients and medical care providers can typically monitor an IMD (e.g., via a wireless connection to an external computer or other electronic device) and assess a patient's current and historical physiological state to identify conditions or predict impending events (e.g., seizures).

In some cases, IMDs may be programmed by, and exchange data with, external devices or equipment controlled by physicians and/or the patient. Communication with such devices and/or equipment may be conducted using a wireless bi-directional communication link with the IMDs. A bi-directional communication link often begins with the broadcast of an advertisement notice by the IMD (e.g., at a predetermined constant frequency based on the wireless protocol). Once an advertisement is received and acknowledged by a compatible external device and/or equipment, a bi-directional communication link may be established between the IMD and the external device and/or equipment. This link may, e.g., allow the patient or a physician to view or modify settings of the IMD, and/or allow for the transfer of sensor logs, configuration profiles, or other data from or to the IMD. Thus, bi-directional communication links provide many advantages and improved convenience for patients with IMDs, including increased customization options for treatment, convenience, and greater access to health-related data.

Unfortunately, in practice these benefits may be limited to some extent by logistical considerations related to wireless communication. For example, current IMDs typically implement the periodic Bluetooth Low Energy (“BLE”) advertisement protocol, in order to allow medical professionals (e.g., using a clinician programmer, “CP” device) and patients (e.g., using a patient remote control, “PRC” device) to scan for and connect to an IMD. In situations where a CP device attempts to connect to an IMD (e.g., an IPG) while a PRC device is already connected, the patient normally must disable the PRC device's Bluetooth connection in order to allow the CP device to connect to the IMD (e.g., the IPG). This approach often results in confusion and unreliable connectivity with IMDs, especially during follow-up clinical visits by patients, potentially wasting IMD battery power, as well as the time of both the patient and medical professional. Accordingly, there exists a need for improves systems and methods for managing wireless communication with IMDs such as IPGs.

### BRIEF SUMMARY OF EXEMPLARY ASPECTS OF THE DISCLOSURE

Ideally, a wireless communication system for IMDs should allow patients and medical professionals to quickly and efficiently establish bi-directional communication between the IMD and one or more external electronic devices or computers. An improved user experience would encourage better patient compliance and utilization of features provided by modern IMDs (e.g., the ability to transfer logged data and to run customized treatment configurations). Moreover, the ability to quickly and reliably establish wireless communication links would help conserve medical resources (e.g., reducing time lost by clinician programmers and other medical professionals simply establishing a bi-directional communication, so that such time may be instead be used to provide patient care). The systems and methods described here address shortcomings of prior systems and in some aspects allow for fast, reliable, and efficient establishment of bi-directional wireless communication with IMDs (e.g., IPGs for treating OSA).

The following presents a simplified summary of several exemplary embodiments in order to provide a basic understanding of the inventions described herein. This summary is not intended as an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In a first general aspect, the disclosure provides a system for managing wireless communication with an implanted medical device, comprising: an implantable medical device (IMD); a first external device, configured to control the IMD using a wireless connection, and to detect the presence of a second external device configured to wirelessly communicate with and control the IMD; wherein the first external device is configured to detect the presence of the second external device based on a) an actual or estimated physical distance between the first external device and the second external device, the first external device and the IMD, and/or the second external device and the IMD; and/or b) geographic location data for the first external device, the second external device, and/or the IMD; wherein the first external device is further configured to terminate the wireless connection used to control the IMD in response to i) detecting the presence of the second external device within a predetermined distance from the first external device and/or the IMD, and/or ii) based upon the geographic location data indicating that the first external device, the second external device, and/or the IMD are located at a predetermined geographic location.

In some aspects, the IMD is an implantable pulse generator (IPG).

In some aspects, the first external device and/or the second external device is an electronic device configured to: a) access or modify one or more settings of the IMD; and/or b) wirelessly receive data from, or transfer data to, the IMD.

In some aspects, the first external device is a patient remote control device configured to be operated by a subject implanted with the IMD, and/or the second external device is a clinician programmer device configured to be operated by a medical professional.

In some aspects, the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

In some aspects, the first external device is configured to terminate the wireless connection used to control the IMD: a) directly; or b) by transmitting a signal to the IMD that triggers the IMD to terminate the wireless connection. In some aspects, the IMD is configured to switch to a mode that only allows establishment of a wireless connection with the second external device for a predefined period of time, after receiving the signal from the first external device triggering the IMD to terminate the wireless connection.

In some aspects, the first external device is configured to terminate the wireless connection used to control the IMD, after receiving a signal from the second external device. In some aspects, the first external device is further configured to switch to a disconnected mode wherein attempts to detect and wirelessly connect to the IMD are disabled, after receiving a signal from the second external device. In some aspects, the signal is received from a cloud-based infrastructure.

In some aspects, the first external device is configured to remain in the disconnected mode: a) for a predetermined period of time, wherein the period of time is optionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or a period of time within a range defined by endpoints selected from any of the foregoing lengths of time; b) until a predetermined signal to exit disconnected mode is received, wirelessly; and/or c) based on manual input entered into an interface of the first external device.

In some aspects, the first external device is further configured to periodically determine geographic location data indicating a geographic location of the first external device, and to switch to a disconnected mode wherein attempts to detect and wirelessly connect to the IMD are disabled, when the geographic location of the first external device is within a predetermined distance from that of a predetermined geographic landmark or location. In some aspects, the geographic landmark or location is a location of a hospital or clinic, optionally stored in a memory of the first external device. In some aspects, the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

In some aspects, the system further comprises an inertial measurement unit (IMU), optionally at least partially contained in a housing of the IMD, and the IMD further comprises a controller configured to detect a signal from the IMU indicative of tactile input provided by a subject implanted with the IMD. In some aspects, the signal comprises a predetermined pattern of taps on or within proximity to the IMU. In some aspects, the controller is configured to terminate the wireless connection with the IMD when the signal is detected. In some aspects, the controller is further configured to cause the IMD to broadcast an advertisement to connect when the signal is detected, and to reject connection attempts made by the first external device for a predetermined period of time. In some aspects, the predetermined period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or a period of time within a range defined by endpoints selected from any of the foregoing lengths of time.

In a second general aspect, the disclosure provides a method for managing wireless communication with an implanted medical device, comprising: providing an implantable medical device (IMD); providing a first external device, configured to control the IMD using a wireless connection, and to detect the presence of a second external device configured to wirelessly communicating with and control the IMD; detecting, by the first external device, the presence of the second external device based on a) an actual or estimated physical distance between the first external device and the second external device, the first external device and the IMD, and/or the second external device and the IMD; and/or b) geographic location data for the first external device, the second external device, and/or the IMD; and terminating the wireless connection between the first external device and the IMD in response to i) detecting the presence of the second external device within a predetermined distance from the first external device and/or the IMD, and/or ii) based upon the geographic location data indicating that the first external device, the second external device, and/or the IMD are located at a predetermined geographic location.

In some aspects of the methods disclosed herein, the first external device is configured to terminate the wireless connection with the IMD: a) directly; or b) by transmitting a signal to the IMD that triggers the IMD to terminate the wireless connection. In some aspects, the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances. In some aspects, the first external device is further configured to periodically determine geographic location data indicating a geographic location of the first external device; and the predetermined condition comprises: the geographic location of the first external device is within a predetermined distance from that of a geographic landmark or location, optionally wherein the geographic landmark is a location of a hospital or clinic, optionally stored in a memory of the first external device. In some aspects, the first external device is a patient remote control device configured to be operated by a subject implanted with the IMD, and/or the second external device is a clinician programmer device configured to be operated by a medical professional. In some aspects, the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

In some aspects, the methods described herein further comprise a step of: receiving a signal from an inertial measurement unit (IMU) indicative of tactile input provided by a subject implanted with the IMD, wherein the signal comprises a predetermined pattern of taps on or within proximity to the IMU; and terminating, by the first external device, the wireless connection with the IMD when the signal is detected.

In still further aspects, the disclosure provides methods capable of performing one or more of the functions of the systems described herein, in any combination. Similarly, it should be understood that the disclosure extends to devices configured to perform any such functions or combinations thereof.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for managing wireless communication with an IMD (in this case, an IPG).

FIG. 2 is a flowchart illustrating aspects of an exemplary method for managing wireless communication with an IMD according to the disclosure.

FIG. 3 is a flowchart illustrating aspects of a second exemplary method for managing wireless communication with an IMD according to the disclosure.

FIG. 4 is a flowchart illustrating aspects of a third exemplary method for managing wireless communication with an IMD according to the disclosure.

FIG. 5 is a flowchart illustrating aspects of a fourth exemplary method for managing wireless communication with an IMD according to the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

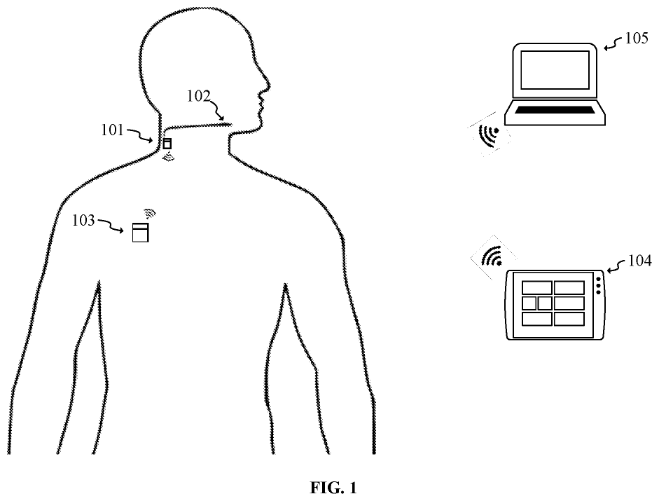

FIG. 1 is a diagram illustrating an exemplary embodiment of a system 100 for managing wireless communication with an IMD (in this case, an IPG 101). As shown in this figure, the IPG 101 controls an electrode 102 and is communicatively-linked to an inertial measurement unit, “IMU” 103). The IPG may be configured to deliver stimulation to a nerve of the subject via the electrode 102 (e.g., to treat OSA), wherein one or more parameters of the stimulation are determined and/or set by a controller comprising memory and a processor. It is envisioned that the IMU 103 may comprise an accelerometer and/or a gyroscope, and be capable of detecting a signal indicative of tactile input provided by the subject implanted with the IPG 101 (e.g., taps or other physical gestures provided by the subject on or in proximity to the IMU 103). In some aspects, the detection of this signal may be a secondary functionality provide by the IMU 103 (the IMU 103 may also be used to detect a signal indicative of respiration, or of the position of or movement by the subject, etc.). It is understood that in some aspects, an IMD such as the IPG 101 shown in this figure, may be configured to deliver stimulation based on sensor data received from one or more internal or external sensors.

As illustrated by FIG. 1, an IMD may be configured to wirelessly communicate with one or more external devices. Such devices include, e.g., a patient remote control device 104 provided to or accessible by the subject (alternatively referred to as a “PRC device” herein), that allows the subject to access, view, and/or control one or more settings or functions of the IMD. The PRC device 104 may comprise a dedicated hardware device, or software executed on a general-purpose computer or other electronic device (e.g., software executed on a tablet or laptop computer) capable of wirelessly communicating with the IMD. The PRC device 104 may be configured, e.g., to allow the subject to access log files comprising historical physiological, stimulation, or other data, to configure settings (e.g., a patient-specific stimulation profile), to enable or disable automatic stimulation (e.g., to place the IMD in a disabled mode while the subject is awake), or any other desired functionality.

In some aspects, the IMD may also be configured to wireless communicate with a separate external device operated by a clinician programmer (alternatively referred to as a “CP” herein). The CP device 105 may provide any or all of the functionality provided by the PRC device 104. In some aspects, the CP device 105 may provide additional functions, settings, or control over the IMD to the CP device 105, which are unavailable to the subject. For example, the CP device 105 may be able to perform diagnostic routines on the IMD or to adjust settings that might result in potential harm to the subject if adjusted by a layperson. Thus, the CP device 105 is understood to be an external electronic device operated by a medical professional and capable of wireless communication with the IMD, that provides an interface to access, view, and/or control one or more settings or functions of the IMD. The CP device 105 will typically be used in a hospital or other clinical setting.

As explained above, one of the shortcomings of modern IMDs is the difficulty associated with establishing a wireless connection to the IMD when a PRC device 104 and a CP device 105 are both in proximity to the IMD. For example, in normal use the IMD will typically be paired with the PRC device 104 because that device serves as the primary means for the subject to control and interact with their IMD. However, there will be times when a medical professional needs to interact with the IMD (to view log files, change settings, etc.), such as when the subject is at a hospital or clinic seeking medical treatment. In that case, the IMD will need to be disconnected from the PRC device 104 and a new connection will need to be established with the CP device 105. The establishment of this connection to the CP device 105 may be delayed due to repeated connection attempts by the PRC device 104, and/or if the IMD is unable to broadcast and advertisement to form a connection due to already having an active connection with the PRC device 104. Accordingly, new protocols are required to allow for an efficient and seamless handoff between these devices.

In some aspects, the systems described herein may comprise a PRC device 104 configured to terminate a previously-established connection with an IMD under predetermined conditions. For example, A PRC device 104 can periodically search for neighboring CP devices 105. Once a CP device 105 is detected within a preset distance (e.g., a 5, 6, 7, 8, 9, 10, 11, 12, 13, 14, 15, 16, 17, 18, 19, or 20-meter range), the PRC device 104 may disconnect itself from IMD (e.g., an IPG 101). This would allow the CP device 105 to detect and connect with the IMD. Alternatively, the PRC device 104 can send a message to the IMD when a CP device 105 is detected, and the IMD can then disconnect itself from the PRC device 104. The IMD may be configured to then allow only CP device 105 connections for a predefined amount of time (e.g., ignoring connection attempts made by the PRC device 104). The PRC device 104 may be configured to perform periodic scans in the background. In some aspects, the PRC device 104 may be configured to scan on Bluetooth and/or Wifi frequencies to accurately detect CP devices 105 and to measure or estimate their proximity.

In some aspects, the CP device 105 may be configured to send a signal to a PRC device 104 that causes the PRC device 104 to disconnect from the IMD (and optionally to stop scanning for a predefined period of time or until a subsequent signal is received). In some aspects, the CP device 105 may be configured to search for an expected IMD (e.g., an IPG). However, if this IMD is not found, the CP device 105 may be configured to send a message to a server capable of contacting the IPG, which may trigger a signal from the server to the IP that causes the IPG to start advertising (i.e., allowing the CP device 105 to establish a connection). In some aspects, the server may alternatively (or additionally) send a signal to the PRC device 104, which would then disconnect itself so that the IPG can start advertising. In some cases, the PRC device 104 may be configured to always attempts to reconnect to the IMD; in such cases, it may be desirable to disable this auto-reconnect functionality during this period (either until a subsequent reactivation sequence is received or for a predetermined amount of time, e.g., 1, 2, 3, 4, 5, 6, 7, 8, 9, 10 or more minutes).

In some aspects, the PRC device 104 may receive geographic location data (e.g., from location services APIs provided by an operating system or application executed on the PRC device 104, provided by the user, or received from the internet or another remote source). The PRC device 104 may be configured to determine the distance between the PRC device 104 and one or more landmarks (e.g., the location of a clinic, hospital, or other location where a CP device 105 may be present). In some aspects, the PRC device 104 may be configured to disconnect from the IMD and/or to stop scanning for connections (or attempting to establish a connection with the IMD) while within a predetermined distance from a landmark, or for a predetermined period of time after coming within proximity to the landmark. For example, once a patient is enrolled in a clinic and the patient's PRC device 104 is associated with a given IMD (e.g., an IPG implanted in the patient), the clinic location may be stored in memory on the PRC device 104, or otherwise made available to the PRC device 104 (e.g., via a web-based query). The PRC device 104 may be configured to regularly determine its current geographic location. If the PRC device 104 comes within a predetermined distance (e.g., 10, 20, 30, 40, 50, 60, 70, 80, 90, or 100 meters) of clinic location, the PRC device 104 may disconnect itself from the IMD, thus allowing a CP device 104 at the clinic to establish a new connection to the IMD.

In some aspects, the IMD may further comprise, or be communicatively linked to, an IMU 103 configured to detect a signal from the IMU 103 indicative of tactile input provided by a subject implanted with the IMD. In some aspects, the signal comprises a predetermined pattern of taps on or within proximity to the IMU 103. For example, the signal may comprise a specific pattern, such as two double-taps within 5 seconds. Any combination of single, double, triple, etc. taps may be used, and the detection window may comprise any number of seconds, e.g., 1, 2, 3, 4, 5, 6, 7, 8, 9, or 10 seconds. In some aspects, a controller of the IMD is configured to terminate a previously-established wireless connection with the PRC device 104 when a predetermined signal (e.g., a specific pattern of taps) is detected. Upon doing so, the IMD would then be able to begin advertising the option to establish a new connection (e.g., allowing a CP device 105 to connect). In some aspects, the IMD may be configured to only accept connection requests from a CP device 105 for a predefined period of time after terminating the connection with the PRC device 104, (e.g., for 30-60 minutes; a typical follow-up visit duration), or until the CP device 105 notifies the IMD that a programming session is complete, in order to prevent the PRC device 104 from automatically reconnecting to the IMD.

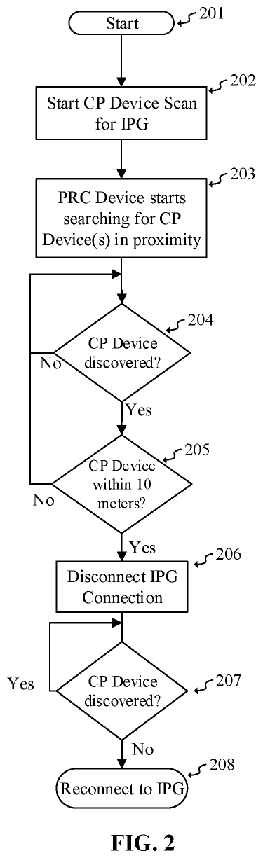

FIGS. 2-5 illustrate exemplary methods for managing wireless communication with an IMD according to the disclosure.

As shown by FIG. 2, an exemplary method for managing wireless communication with an implanted medical device may begin (201) with a CP device 105 scanning for an implantable device, such as an IPG 101 (202). Subsequently, or in parallel, a PRC device 104 may start searching for CP devices 105 in proximity to the PRC device 104 (203). If a CP device 105 is discovered (204), the PRC device 104 may then determine whether the CP device 104 is within a predetermined distance from the PRC device 104 (205). As explained herein, the proximity range or threshold for this distance may comprise any geographic distance desired for a given implementation, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances. If the CP device 105 is determined to be within the predetermined range, the PRC device 104 may be configured to disconnect a current wireless connection established with the IPG 101 (206). The wireless connection may be, e.g., a Bluetooth connection (e.g., BLE), a wireless connection (e.g., 802.11 a/b/g/n/ac). Thereafter, the PRC device 104 may be configured to engage in one or more optional activities as described herein. For example, as shown by this figure it may be configured to continue scanning for CP devices 105 and, if one is discovered (207), it may be configured to reestablish a connection with the IPG 101 (208).

In other aspects, the PRC device 104 may be configured to perform other or additional functions. For example, upon detection of a CP device 105, the PRC device 104 may be configured to stop scanning for a predetermined amount of time, or to stop attempting to establish a wireless connection with the IPG 101 for a predetermined time. In some aspects, the PRC device 104 may further be configured to distinguish between different CP devices 105 (e.g., based on a serial number or other unique identified associated with different CP devices 105). In some aspects, the PRC device 104 may be configured to perform any of the functions described herein upon (or only when) a specific CP device 105 is detected within a predetermined distance (e.g., it may be useful to have a PRC device 104 recognize a CP device 105 used by the subject's physician, rather than other CP devices 105 that may be present at a hospital or other medical facility).

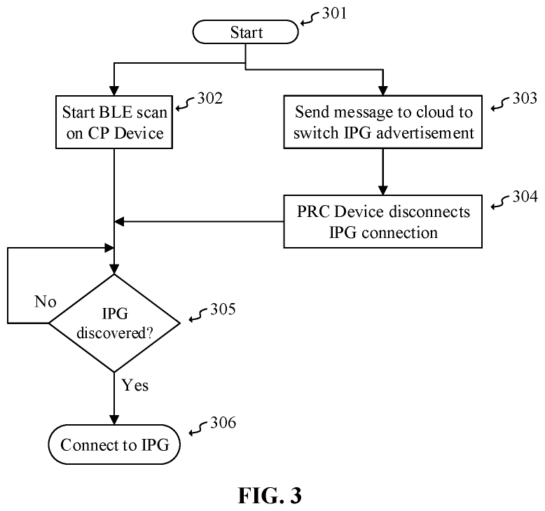

As shown by FIG. 3, another exemplary method for managing wireless communication with an implanted medical device may begin (301) with a CP device 105 starting to scan (using the BLE protocol, or another wireless protocol) to detect an IPG 101 in proximity to the CP device 105 (302). In parallel, the CP device 105 may optionally be configured to transmit a message to the IPG 101 via a cloud-based infrastructure to cause the IPG 101 to switch advertising its availability for a connection (303). For example, the IPG 101 may receive a signal via the cloud-based infrastructure causing the IPG 101 to terminate a previously-established connection with a PRC device 104, or to terminate all current wireless connections. Alternatively, the IPG 101 may receive a signal via the cloud-based infrastructure causing the IPG 101 to begin advertising its availability to establish a wireless connection, after previously having been in a state where it was not actively broadcasting an advertisement to connect. Thereafter, a PRC device 104 may disconnect from a wireless connection previously-established with the IPG 101 (304). In either case, the CP device 105 may then detect an IPG 101 that is available to establish a wireless connection (e.g., via BLE) (305) and to connection to the IPG 101 (306).

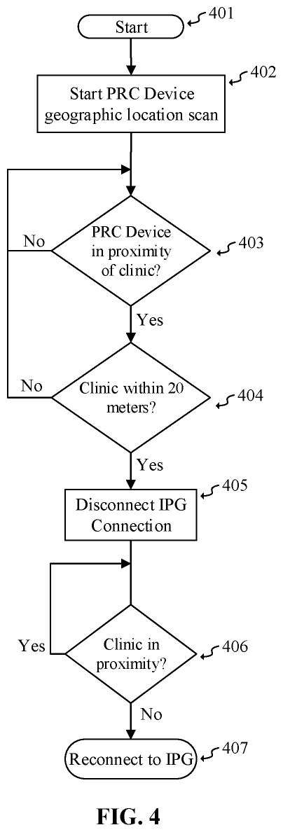

As shown by FIG. 4, another exemplary method for managing wireless communication with an implanted medical device may start (401) with a PRC device 104 obtaining geographic location data. In this case, the PRC device 104 is configured to perform a wireless scan to obtain geographic location data (402). For example, the PRC device 104 may be configured to detect nearby wireless networks and to recognize one or more access point SSIDs (or other signals) as an indication of being within physical proximity to a clinic, hospital, medical facility (or another location or type of facility). In other aspects, the PRC device 104 may be configured to determine its geographic location using a GPS sensor incorporated into the PRC device 104, or to obtain data indicative of its geographic location from an external device (a paired cell phone, smart phone, smart watch, computer, etc.). When the PRC device 104 determines that it is within a predetermined distance from a predetermined location, e.g., a clinic (404), it may be configured to disconnect a previously-established wireless connection with an IPG 101 (405). Thereafter, the PRC device 104 may be configured to continue obtaining geographic location data in order to determine whether the PRC device 104 is still within proximity to the predetermined location, and if not, the PRC device 104 may then proceed to reconnect to the IPG 101 (407).

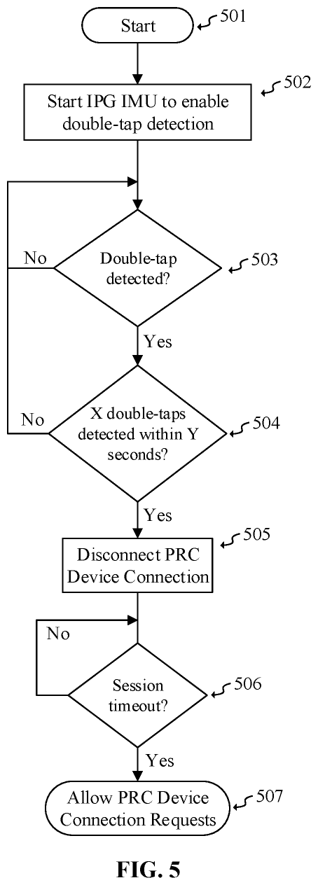

As shown by FIG. 5, another exemplary method for managing wireless communication with an implanted medical device may start (401) with an IMU 103 of an IPG 101 being enabled to detect a signal indicative of a tactile input (e.g., a double-tap by the subject). A controller of the IPG 101 (optionally part of the IMU 103) may be configured to determine whether the signal detected by the IMU 103 satisfies one or more predetermined patterns. As explained herein, a pattern of tactile input may comprise, e.g., 1, 2, 3, 4, or 5 single-taps, double-taps, triple-taps, etc., or any combination thereof (e.g., 2 double-taps, or a double-tap and a triple-tap). The controller may also be configured to take into account a timeframe or threshold of time when determining whether a pattern of tactile input has been satisfied (e.g., the tactile input signal may be evaluated over a period of 1, 2, 3, 4, 5, 6, 7, 8, 9, or 10 seconds, or within a range defined by any pair of the foregoing lengths of time). In some aspects, the controller may further be configured to determine whether the tactile input resulted from one or more taps within a predetermined distance from the IMU 103 (e.g., within 1, 2, 3, 4, 5, 6, 7, 8, 9, 10, 11, 12, 13, 14, 15, 16, 17, 18, 19, or 20 cm), allowing for improved recognition of false positives. In any case, if a predetermined pattern of tactile input is detected (e.g., X double-taps within Y seconds as shown in this FIG. 504), the IPG 101 may be configured to disconnect from a previously-established connection with a PRC device 104 (505). At this stage, the IPG 101 may then be paired with a CP device 105, another device, or may be remain in an unpaired state. The IPG 101 may be configured not to allow a new connection with a PRC device 104 for a predetermined time (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or a period of time within a range defined by endpoints selected from any of the foregoing lengths of time. After this time period passes (506), the IPG may be configured to allow a new connection to be established with a PRC device 104.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A system for managing wireless communication with an implanted medical device, comprising:
an implantable medical device (“IMD”);
a first external device, configured to control the IMD using a wireless connection, and to detect the presence of a second external device configured to wirelessly communicate with and control the IMD;
wherein the first external device is configured to detect the presence of the second external device based on
a) an actual or estimated physical distance between the first external device and the second external device, the first external device and the IMD, and/or the second external device and the IMD; and/or
b) geographic location data for the first external device, the second external device, and/or the IMD;
wherein the first external device is further configured to terminate the wireless connection used to control the IMD in response to i) detecting the presence of the second external device within a predetermined distance from the first external device and/or the IMD, and/or ii) based upon the geographic location data indicating that the first external device, the second external device, and/or the IMD are located at a predetermined geographic location.

2. The system of claim 1, wherein the IMD is an implantable pulse generator (“IPG”).

3. The system of claim 1, wherein the first external device and/or the second external device is an electronic device configured to:
a) access or modify one or more settings of the IMD; and/or
b) wirelessly receive data from, or transfer data to, the IMD.

4. The system of claim 1, wherein the first external device is a patient remote control device configured to be operated by a subject implanted with the IMD, and/or the second external device is a clinician programmer device configured to be operated by a medical professional.

5. The system of claim 1, wherein the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

6. The system of claim 1, wherein the first external device is configured to terminate the wireless connection used to control the IMD:
a) directly; or
b) by transmitting a signal to the IMD that triggers the IMD to terminate the wireless connection.

7. The system of claim 6, wherein the IMD is configured to switch to a mode that only allows establishment of a wireless connection with the second external device for a predefined period of time, after receiving the signal from the first external device triggering the IMD to terminate the wireless connection.

8. The system of claim 1, wherein the first external device is configured to terminate the wireless connection used to control the IMD, after receiving a signal from the second external device.

9. The system of claim 8, wherein the first external device is further configured to switch to a disconnected mode wherein attempts to detect and wirelessly connect to the IMD are disabled, after receiving a signal from the second external device.

10. The system of claim 9, wherein the first external device is configured to remain in the disconnected mode:
a) for a predetermined period of time, wherein the period of time is optionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or a period of time within a range defined by endpoints selected from any of the foregoing lengths of time;
b) until a predetermined signal to exit disconnected mode is received, wirelessly; and/or
c) based on manual input entered into an interface of the first external device.

11. The system of claim 8, wherein the first external device received the signal from a cloud-based infrastructure.

12. The system of claim 1, wherein the first external device is further configured to periodically determine geographic location data indicating a geographic location of the first external device, and to switch to a disconnected mode wherein attempts to detect and wirelessly connect to the IMD are disabled, when the geographic location of the first external device is within a predetermined distance from that of a predetermined geographic landmark or location.

13. The system of claim 12, wherein the geographic landmark or location is a location of a hospital or clinic, optionally stored in a memory of the first external device.

14. The system of claim 12, wherein the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

15. The system of claim 1, wherein the system further comprises an inertial measurement unit (“IMU”), optionally at least partially contained in a housing of the IMD, and
wherein the IMD further comprises a controller configured to detect a signal from the IMU indicative of tactile input provided by a subject implanted with the IMD.

16. The system of claim 15, wherein the signal comprises a predetermined pattern of taps on or within proximity to the IMU.

17. The system of claim 15, wherein the controller is configured to terminate the wireless connection with the IMD when the signal is detected.

18. The system of claim 17, wherein the controller is further configured to cause the IMD to broadcast an advertisement to connect when the signal is detected, and to reject connection attempts made by the first external device for a predetermined period of time.

19. The system of claim 18, wherein the predetermined period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or a period of time within a range defined by endpoints selected from any of the foregoing lengths of time.

20. A method for managing wireless communication with an implanted medical device, comprising:
providing an implantable medical device (“IMD”);
providing a first external device, configured to control the IMD using a wireless connection, and to detect the presence of a second external device configured to wirelessly communicating with and control the IMD;
detecting, by the first external device, the presence of the second external device based on
a) an actual or estimated physical distance between the first external device and the second external device, the first external device and the IMD, and/or the second external device and the IMD; and/or
b) geographic location data for the first external device, the second external device, and/or the IMD; and
terminating the wireless connection between the first external device and the IMD in response to i) detecting the presence of the second external device within a predetermined distance from the first external device and/or the IMD, and/or ii) based upon the geographic location data indicating that the first external device, the second external device, and/or the IMD are located at a predetermined geographic location.

21. The method of claim 20, wherein the first external device is configured to terminate the wireless connection with the IMD:
a) directly; or
b) by transmitting a signal to the IMD that triggers the IMD to terminate the wireless connection.

22. The method of claim 20, wherein the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

23. The method of claim 22, wherein the first external device is further configured to periodically determine geographic location data indicating a geographic location of the first external device; and the predetermined condition comprises:
the geographic location of the first external device is within a predetermined distance from that of a geographic landmark or location, optionally wherein the geographic landmark is a location of a hospital or clinic, optionally stored in a memory of the first external device.

24. The method of claim 20, wherein the first external device is a patient remote control device configured to be operated by a subject implanted with the IMD, and/or the second external device is a clinician programmer device configured to be operated by a medical professional.

25. The method of claim 23, wherein the predetermined distance is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meters, or a distance within a range defined by endpoints selected from any of the foregoing distances.

26. The method of claim 23, further comprising
receiving a signal from an inertial measurement unit (“IMU”) indicative of tactile input provided by a subject implanted with the IMD, wherein the signal comprises a predetermined pattern of taps on or within proximity to the IMU; and
terminating, by the first external device, the wireless connection with the IMD when the signal is detected.
